(19) United States
(12) Patent Application Publication
(10) Pub. No.: US 2018/0092797 A1
(43) Pub. Date: Apr. 5, 2018

(54) Mechanical Compression Device For Treating Or Preventing Deep Vein Thrombosis And Associated Complications

(71) Applicants: Robert N. Uzzo, Ambler, PA (US); Institute For Cancer Research d/b/a The Research Institute of Fox Chase Cancer Center, Philadelphia, PA (US)

(72) Inventors: Robert G. Uzzo, Philadelphia, PA (US); Robert N. Uzzo, Ambler, PA (US)

(21) Appl. No.: 15/722,077

(22) Filed: Oct. 2, 2017

Related U.S. Application Data
(60) Provisional application No. 62/403,236, filed on Oct. 3, 2016.

(51) Int. Cl. A61H 9/00; A61F 13/08

(52) U.S. Cl. CPC A61H 9/0078; A47G 25/905; A61H 2209/00; A61F 13/08

(57) ABSTRACT

A portable, mechanical compression device for preventing or treating deep vein thrombosis and extremity edema is provided herein. The device includes a frame that is placed in whole or in part around a limb, and a plurality of compression units that compress the limb, thereby mechanically facilitating circulation. A user (e.g., wearer or helper) moves the device along the limb, thereby moving the point of compression along the limb in the direction of the movement of the device, facilitating blood flow through the limb, thereby lessening the chance of a clot formation within the deep veins of the limb and decreasing stasis of fluid or blood in the limb.

### CROSS-REFERENCE TO RELATED APPLICATIONS

This application claims priority to U.S. Application No. 62/403,236, filed Oct. 3, 2016, which is incorporated herein by reference in its entirety.

### FIELD

The present disclosure relates generally to venous circulation. In particular, the present disclosure relates to devices that can be used to reduce pooling and clotting of blood within the veins of the limbs of the body, by providing sequential compression of the limbs to squeeze blood and lymph out of the compressed area. Movement of the device along the limbs facilitates blood displacement from and flow through the deep veins of the limbs.

### BACKGROUND

Various publications, including patents, published applications, technical articles and scholarly articles are cited throughout the specification. Each of these cited publications is incorporated herein by reference in its entirety and for all purposes.

Deep vein thrombosis is a condition that occurs when a blot clot forms in one or more of the deep veins of the body. Most typically, the clot forms in the deep veins of the lower legs. Deep veins are those that are located well within the interior of the limbs. Deep vein thrombosis is often caused by immobility of the limbs, for example, by prolonged sitting, or by surgery or trauma. Multiple risk factors for deep vein thrombosis include pregnancy, obesity, immobility, and genetic predispositions. The formation of the clot can cause pain and swelling of the affected limb. More seriously, the clot can dislodge and cause an embolism, such as a pulmonary embolism (blood clot lodged in the lungs). If untreated, a deep vein thrombosis can lead to damage of the limbs, or even death.

For hospitalized patients, electrical pneumatic devices are often employed to help mitigate against the formation of deep vein thrombosis. These devices are attached to the limbs, and are inflated and deflated to pressurize the tissue and facilitate blood flow. These devices are not portable and require electrical current. Hospitalized patients also may receive pharmacological prophylaxis to prevent DVTs, but not all patients can receive them due to other conditions and predispositions.

For individuals who will be traveling and seated for extended periods of time, it is recommended that they get up and walk periodically, or perform certain movements of the limbs and extremities. Specialized garments such as static compression stockings may also be worn to squeeze the limbs to reduce blood pooling, though such stockings do not facilitate blood flow.

Nevertheless, pneumatic devices are not typically portable, particularly for use on airplanes or trains. In addition, in confined spaces such as the middle seat of an airplane, it may not always be possible to continually get up and walk. Accordingly, there is a need for portable devices, that can be used to prevent the formation of deep vein thrombosis and to treat existing deep vein thrombosis or to treat developing deep vein thrombosis. Moreover, there is a need for a non-pharmacological treatment for outpatients who maintain a risk of DVT.

### SUMMARY

The present disclosure provides devices for compressing a limb, including the muscles of the limb, to facilitate blood flow through the deep veins of the limb. The devices generally comprise a frame, which can comprise an open configuration or a closed configuration. The closed configuration can be a tube-like or sleeve-like configuration. The frame comprises a plurality of compression units on the internal surfaces of the frame. The compression units can comprise one or more balls, wheels, rollers, or a ball transfer unit. The ball transfer unit can comprise ball bearings or a spring. The frame can be unitary (i.e., a single piece) or may comprise a plurality of interconnected segments, which are connected by one or more pivotal linkages that allow the segments to move relative to each other and thereby allow the frame to expand and contract. The pivotal linkages can comprise an elastic material (e.g., natural or synthetic rubber, silicone, polymers, etc.) and/or a spring. In some embodiments, the device can include a mechanical (e.g., hand pumped) pneumatic device.

The device can further comprise a lower leash having elasticity, which leash is connected at one end to the frame, and is connected at the other end to a patch or strap for tethering the frame to a limb. The device can further comprise one or more upper leashes that are connected at one end to the frame, and at the other end to one or more handles.

The present disclosure also provides methods of displacing fluid from and flow through a deep vein of a limb of a subject comprising: attaching any of the devices described herein to the limb, thereby compressing the limb; and moving the device along the limb, thereby displacing fluid from and flow through the deep vein of the limb. Movement of the frame along the limb and, in the process, compressing the limb, decreases stasis of fluid or blood in the limb, and facilitates blood flow through the limb. The repetitive and sequential compression and movement treats and prevents deep vein thrombosis, edema, and associated complications.

### BRIEF DESCRIPTION OF THE DRAWINGS

The present disclosure can be understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1A shows a view of a unitary frame of the compression device in an open, cup-like configuration, having a plurality of compression units.

FIG. 1B shows a view of a segmented frame of the compression device in an open, cup-like configuration, having pivotal linkages between the segments, and a plurality of compression units.

FIG. 1C shows a view of a segmented frame of the compression device in a tube-like or sleeve-like configuration.

FIG. 1D shows a partially open frame of the compression device in a tube-like or sleeve-like configuration.

FIG. 1E shows an open frame (top; above the arrow) with closures, and also shows a closed frame (bottom; below the arrow) in which the open frame has been closed into a tube-like or sleeve-like configuration, and secured closed with the closures. The arrow represents the action of closing the open frame into the closed, tube-like configuration.

FIG. 2A shows a compression unit with a ball transfer configuration.

FIG. 2B shows a cut-away view of a compression unit with a spring operably connected to the ball.

FIG. 2C shows a compression unit with a wheel roller configuration.

FIG. 2D shows a compression unit with a cylindrical roller configuration.

FIG. 3A shows a compression device having two handles and a foot strap, with each leash integral with the frame of the device.

FIG. 3B shows a compression device having two handles and a foot strap, with each leash connected to the frame via clips attached to the connector.

FIG. 3C shows a compression device having two handles and a foot strap, with each handle integral with the frame.

FIG. 4A shows a compression device attached to the calf of a leg, and secured to the foot via a foot strap. The lower leash is taut as the frame has been pulled to the top of the calf along the axis x.

FIG. 4B shows a compression device attached to the calf of a leg, and secured to the foot via a foot strap. The lower leash is slack, as the elasticity of the lower leash has pulled the frame to the bottom of the calf along the axis x.

FIG. 4C shows an open, cup-like configuration of a compression device attached to the calf of a leg, and secured to the foot via a foot strap. The lower leash is slack, as the elasticity of the lower leash has pulled the frame to the bottom of the calf along the axis x.

### DESCRIPTION OF EMBODIMENTS

Various terms relating to aspects of the present disclosure are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

The present disclosure provides a device 100 for compressing limbs. The limb can be any limb of a mammal, particularly a human, such as an arm or a leg. In some embodiments, the limb is a leg. In some embodiments, the limb is the lower portion of a leg (i.e., blow the knee). The device can be used in the treatment and/or prevention of deep vein thrombosis, and to treat and/or prevent edema (i.e., swelling) that is associated with deep vein thrombosis. The device can also be used to treat and/or prevent edema associated with chronic extremity edema or stasis. In some embodiments, the device is manually controlled by the user or other caregiver, without the need for or controlled by electronic or pneumatic actuation. In some embodiments, the user or other caregiver can control certain aspects of the device by using electronic or mechanical pneumatic actuation.

As described in more detail below, the device is placed around a limb, typically a leg, and moved up and down the limb. The device compresses tissue in the limb (e.g., the muscles in the limb) to help push blood through the limb, thereby preventing blood from pooling within the veins of the limb. The embodiments described herein provide a mechanism whereby venous return is increased with variable pressure. In addition, the embodiments described herein provide a dynamic compression of a limb.

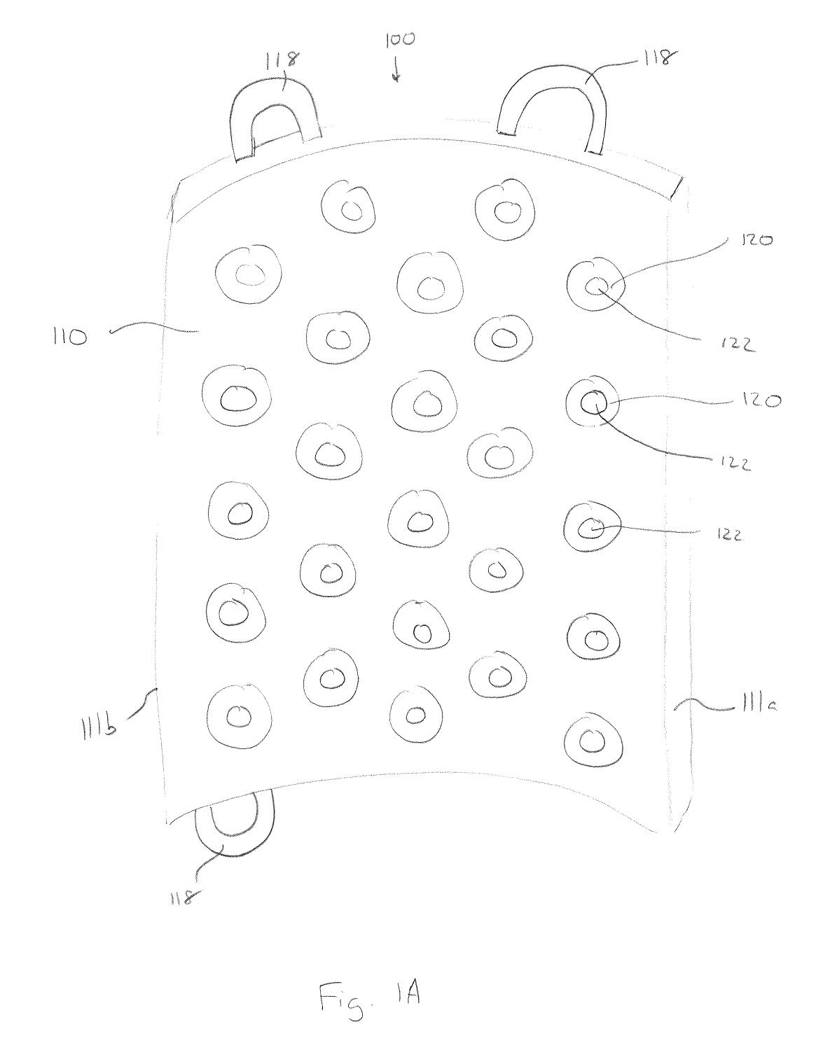

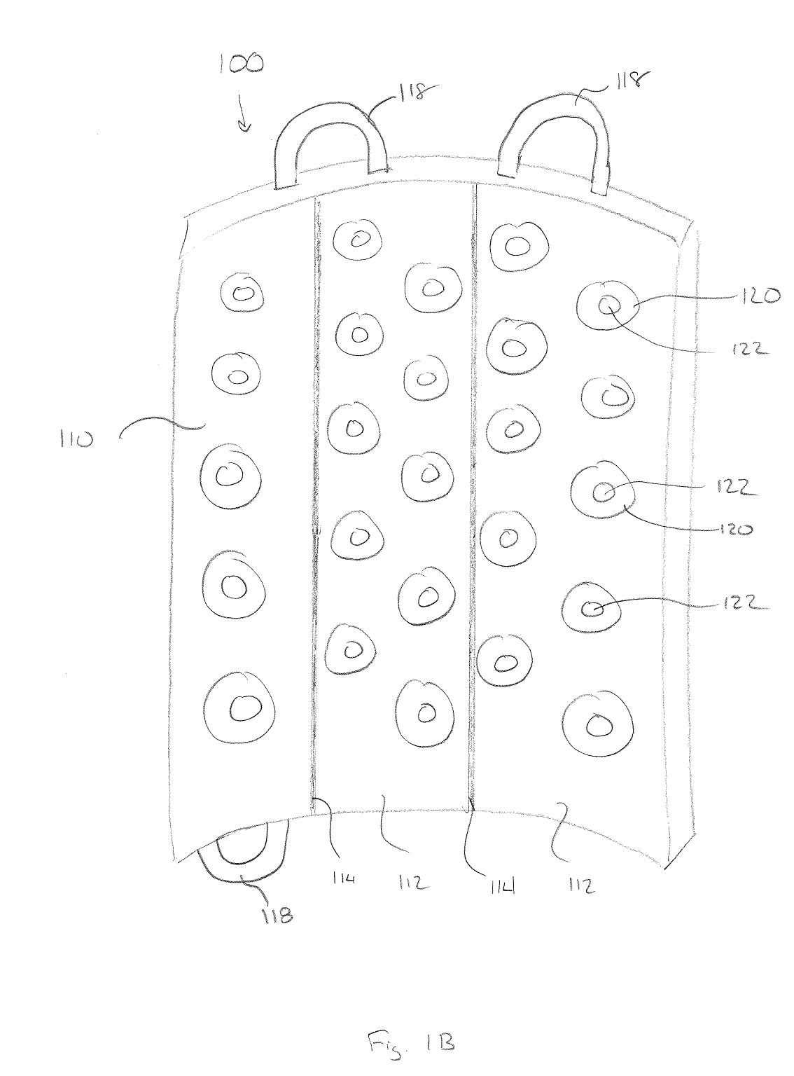

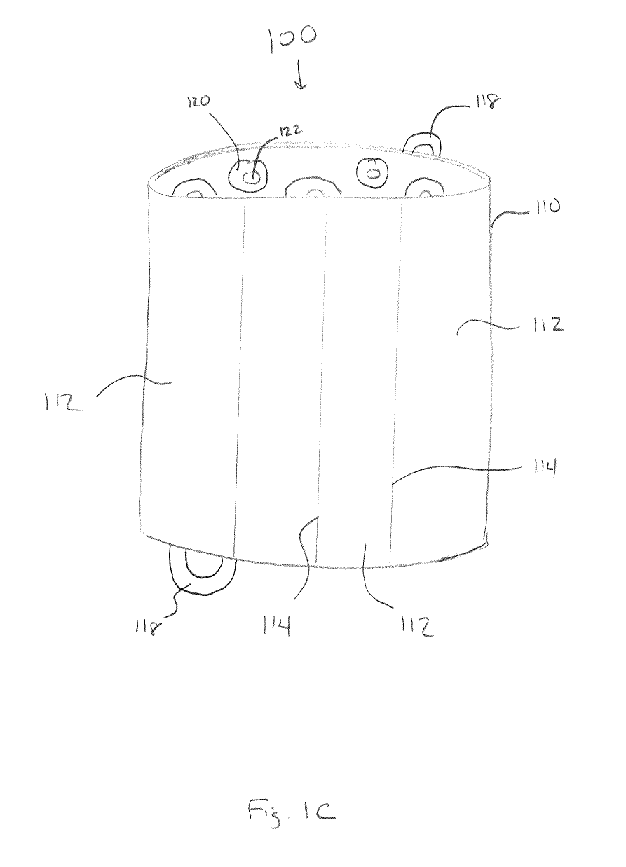

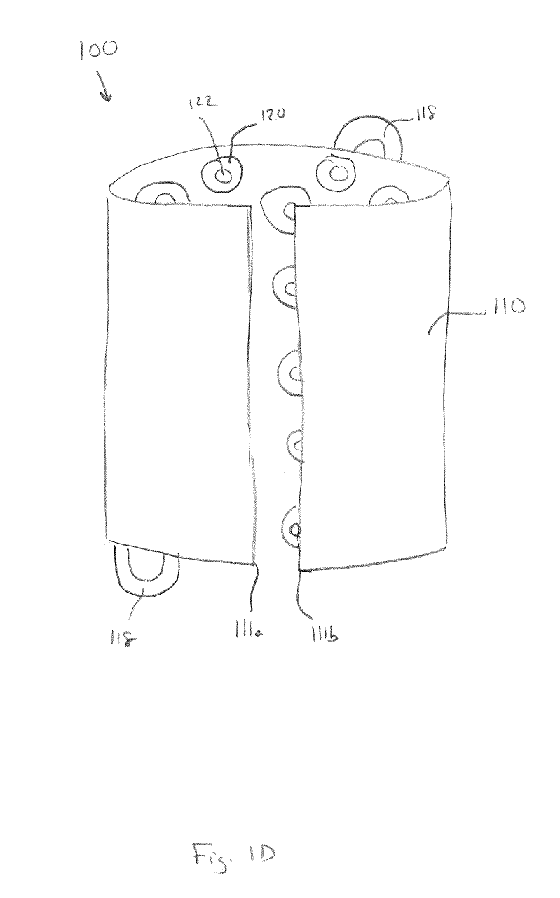

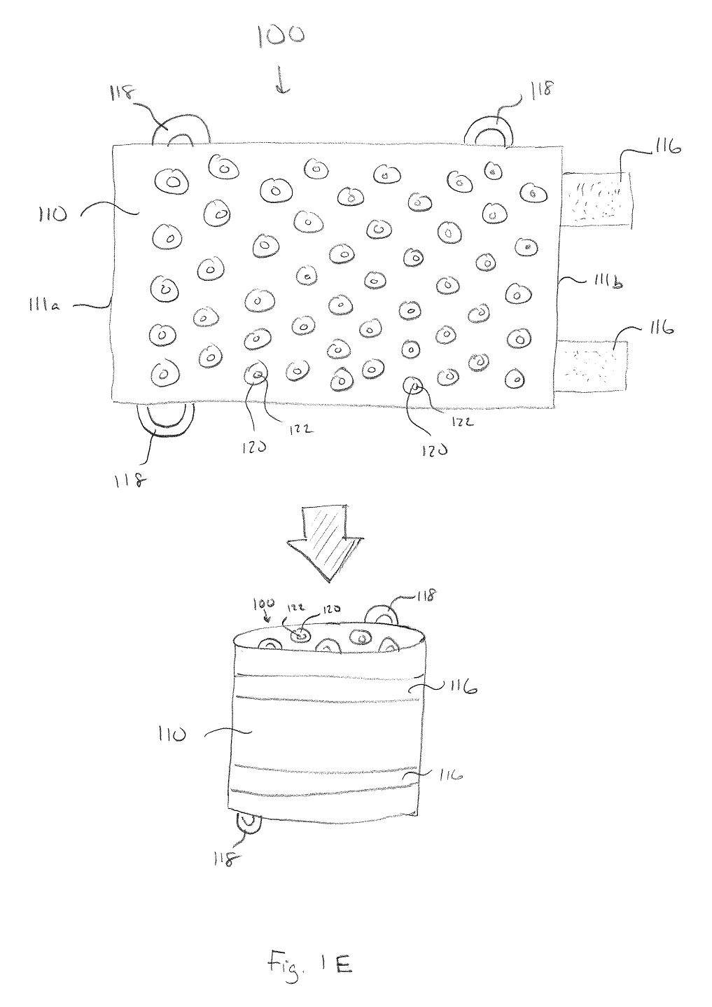

Referring to FIGS. 1A, 1B, 1C, 1D, and 1E, the device 100 comprises a frame 110. In some embodiments, the frame 110 may be rigid, partially flexible, or fully flexible and, in such embodiments, can comprise a shape or form that substantially conforms to the shape and contours of the body limb around which it is placed. In some embodiments, the frame 110 is sufficiently flexible such that it will conform to the shape and contours of the body limb around which it is placed. The frame 110 can comprise any suitable material, including plastic or polymer, metal, ceramic, rubber, elastic, wood, fabric, or any combination thereof. The frame 110 can comprise a mesh or net. In some embodiments, the frame 110 provides suitable compression without the need for any additional compression. In some embodiments, the frame 110 can be inserted or incorporated into a textile or fabric or material that is worn by the individual, by way of example, but not of limitation, such as a sock, stocking, sleeve, shirt, band, legging, or pants.

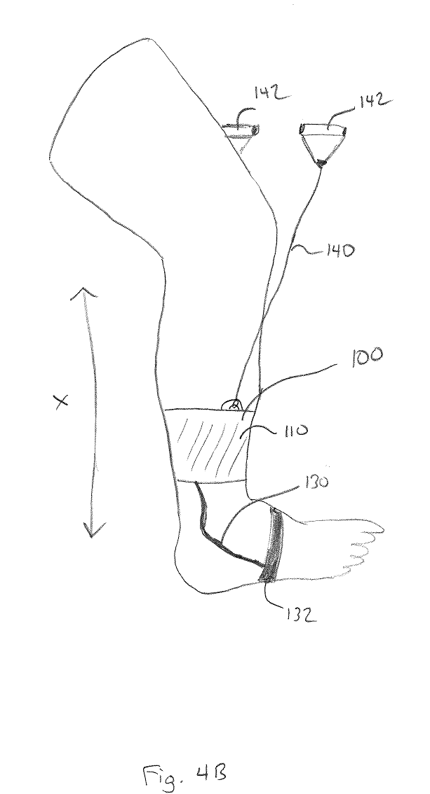

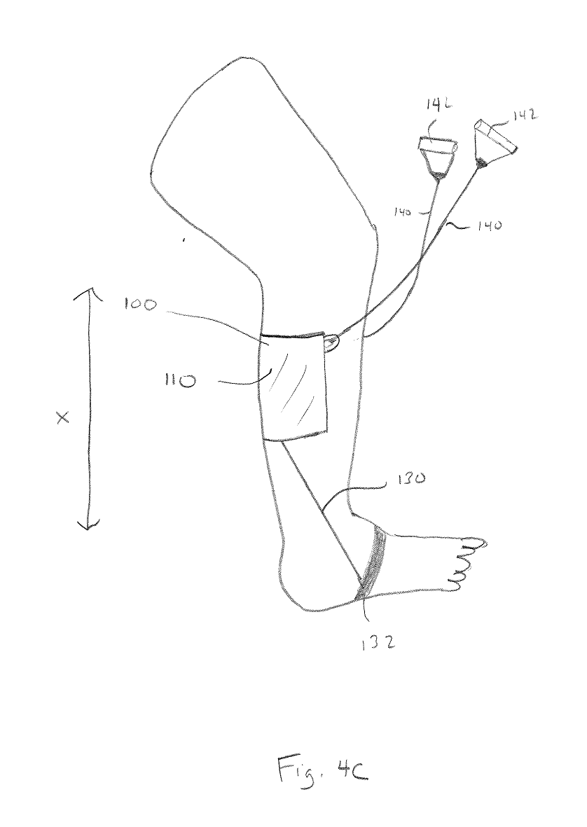

The frame 110 may comprise several configurations. For example, the frame 110 may be a unitary piece (see, FIG. 1A), or may comprise a plurality of interconnected segments 112 (see, FIG. 1B). In some embodiments, frames of this configuration may be cup-like such that it does not completely encircle the limb. In each of these configurations, the frame does not completely wrap around the limb. As shown in FIG. 1A, for example, the frames of this type of configuration have ends 111a and 111b. For example, devices of these configurations may be applied to the back of the calf of the lower leg (see, for example, FIG. 4C).

In some embodiments, to facilitate compression of the limb, the frame 110 can comprise a plurality of interconnected segments 112 that are joined together with a pivotal linkage 114 that allows the segments 112 to move relative to each other (see, for example, FIGS. 1B and 1C), to better conform to the shape and contours of different limbs, including the capacity to accommodate limbs from different sized patients, and also to conform to the changing shape and contours of the limb as the frame 110 of the device 100 is moved along the limb, as well as adjust to limbs that have swollen. In some embodiments, the pivotal linkage 114 imparts the capacity of the frame 110 to expand and contract, with contraction providing inward pressure to ensure a tight fit around the limb, as well as to compress the muscles of the limb. The expansion of the frame 110 via the pivotal linkage 114 allows the frame 110 to be positioned onto the limb (e.g., to be moved over the foot and ankle), as well as to conform to the outwardly tapering shape of the calf and other contours of the limb.

In some embodiments, to enhance the compression forces, the diameter or width of the interior of the frame 110 tube can be smaller than the diameter or width of the limb on which the frame 110 is placed. Thus, once placed on the limb, the frame's 110 narrower diameter or width exerts inward pressure against the limb, thereby compressing the limb, and/or rolling the musculature of the limb. When the frame 110 comprises a plurality of interconnected segments 112 joined together with a pivotal linkage 114 and is placed on a limb, the elasticity of the pivotal linkages 114 contracts to pull the segments 112 inward to assume the narrower diameter or width of the interior of the frame 110 tube and, in so doing, causes the exertion of inward pressure against the limb, thereby compressing the limb.

The pivotal linkages 114 allow the segments 112 of the frame 110 to flex, pivot, deform, expand, and contract relative to other segments 112, establishing elasticity and resilience of the frame 110. The movement provided by the pivotal linkages may be longitudinal, transverse, or torsional. Movement maybe uniaxial, biaxial, triaxial, quadaxial, or pentaxial. The pivotal linkage 114 may comprise a reverse motion linkage, push-pull linkage, parallel motion linkage, bell crank linkage, crank and slider linkage, worm gear, scissor linkage, or any other suitable linkage that provides appropriate motion. The pivotal linkages 114 may comprise a spring or elastic band in some embodiments.

Other configurations of the frame 110 include, for example, a tube-like or sleeve-like configuration (see, FIG. 1C). The frame 110 can be uniform (i.e., without segments; not shown) or can also comprises segments 112 (as depicted in FIG. 1C). Devices having this frame configuration completely wrap around the limb. In some embodiments, devices having this frame configuration can be applied to the limb like a sock or sweat-band.

Other configurations of the frame 110 include, for example, an open configuration (see, FIG. 1D), which is similar to the tube-like or sleeve-like configuration except having a gap therein, such that it does not completely encircle the limb. As shown in FIG. 1D, for example, the frames of this type of configuration have ends 111a and 111b. In some embodiments, this open configuration frame can have one or more closures 116, which can close the frame 110 by bringing end 111a adjacent to end 111b (see, FIG. 1E). By way of example, but not of limitation, the one or more closures 116 can comprise one or more ties, straps, clasps, clips, buckles, snaps, or hook and loop closures such as VELCRO® (Velcro Industries B.V.). By engaging the closures, this open configuration frame can encircle the limb.

The frame 110 further comprises a plurality of compression units 120 (see, FIGS. 1A through 1E). The number of compression units 120 is not critical and can vary, for example, according to the needs of the user or the size of the frame 110. In some embodiments, each compression unit 120, though affixed to the frame 110, has at least a portion that moves or rotates, which portion is placed in contact with the limb. Thus, when the user (or caretaker, trainer, therapist, practitioner, assistant, associate, etc.) moves the frame 110, the compression units 120 move or rotate, thus allowing the frame 110 to more easily move up and down the limb.

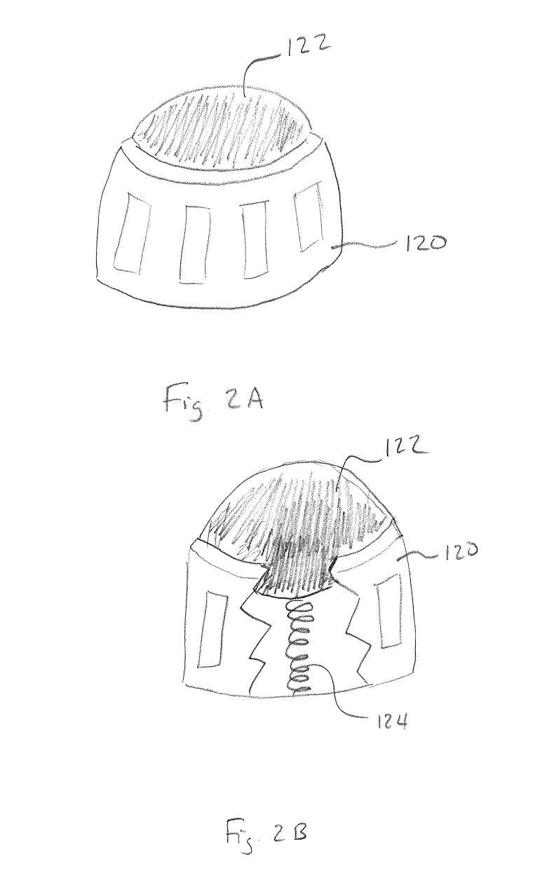

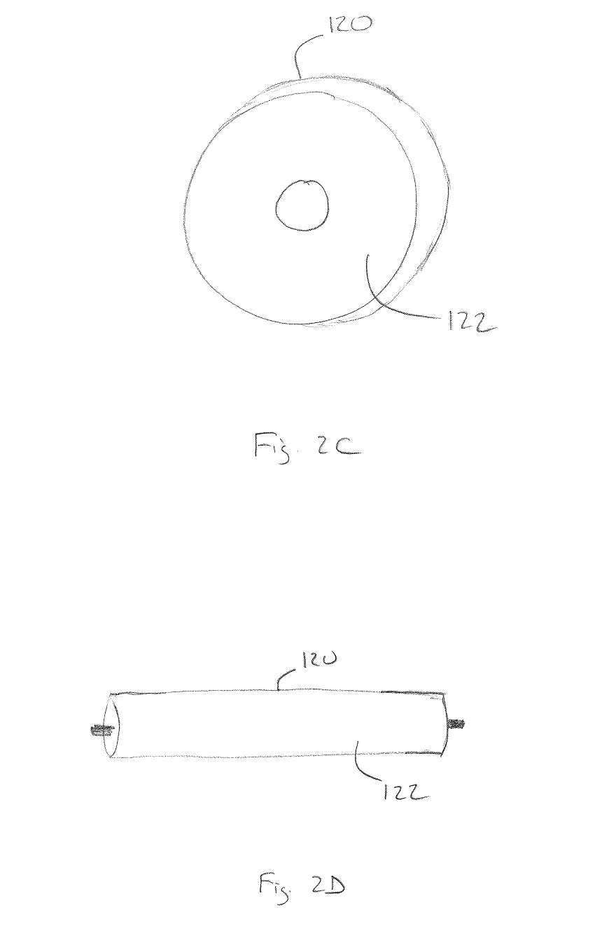

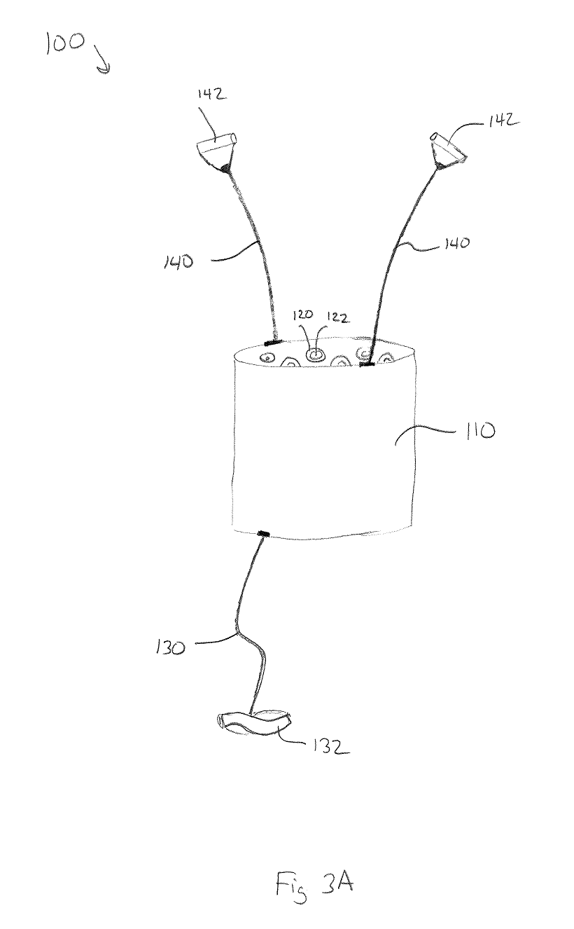

Each compression unit 120 can comprise one or more rollers 122, balls 122, wheels 122, cylinders 122, bearings 122, or other structure capable of movement or rotation (see, FIGS. 1A through 1E, and FIGS. 2A through 2D). In some embodiments, each compression unit 120 is a ball transfer unit comprising a ball 122 (see, FIG. 2A). The one or more rollers 122, balls 122, wheels 122, cylinders 122, bearings 122, or other structure capable of movement or rotation, rotate or move when the user moves the frame 110, thereby facilitating movement over skin or clothing.

The compression units 120 are subject to the inwardly-directed compression forces exerted by the structure of the frame 110. Thus, each compression unit 120 compresses inwardly against the limb, further compressing the limb in the direction of venous return. The compression force exerted by each of the compression units 120 and the frame 110 is continually applied, owing to the elasticity of the frame. As the user (or caretaker, trainer, therapist, practitioner, assistant, associate, etc.) moves the frame 110 along the limb, the compression force is thereby continually applied against the limb where the frame 110 and the compression units 120 are in contact with the limb.

In some embodiments, one or more of the compression units 120 comprise a spring 124 (see, FIG. 2B). The spring 124 may exert outward force against the one or more rollers 122, balls 122, wheels 122, cylinders 122, bearings 122, or other structure capable of movement or rotation, to enhance the compression force of the compression unit 120 against the limb. The spring 124 can also allow each of the one or more rollers 122, balls 122, wheels 122, cylinders 122, bearings 122, or other structure capable of movement or rotation to flex inward toward the frame, in order to better conform to the shape and contours of the limb as the frame 110 is moved along the limb, as well as to provide better comfort to the user. The spring 124 can, thus, further facilitate movement of the frame 110 along the limb and allow the compression forces being applied to the limb to be forgiving when needed to avoid undue pain or discomfort during use.

In some embodiments, the frame 110 can comprise an additional expandable impermeable bladder connected to an electrical or pneumatic pump or control unit through tubing for introducing gas (e.g., air) or liquid (e.g., water) to apply additional compression to the limb. In some embodiments, the liquid or air can be heated or chilled. In some embodiments, the frame 110 can comprise one or more impermeable bladder segments. The tubing for introducing gas or liquid to the bladder can comprise one or more one-way, two-way or three-way valves, as well as a pressure transducer downstream of the valves. In some embodiments, the user of the device or caretaker can control the amount of additional compression (or decompression) by adjusting the electrical or mechanical pneumatic pump. In some embodiments, a mechanical pneumatic pump is adjusted. In some embodiments, the plurality of interconnected segments 112 can comprise an expandable impermeable bladder connected to an electrical or pneumatic pump or control unit through tubing for introducing gas (e.g., air) or liquid (e.g., water) to apply compression to the limb.

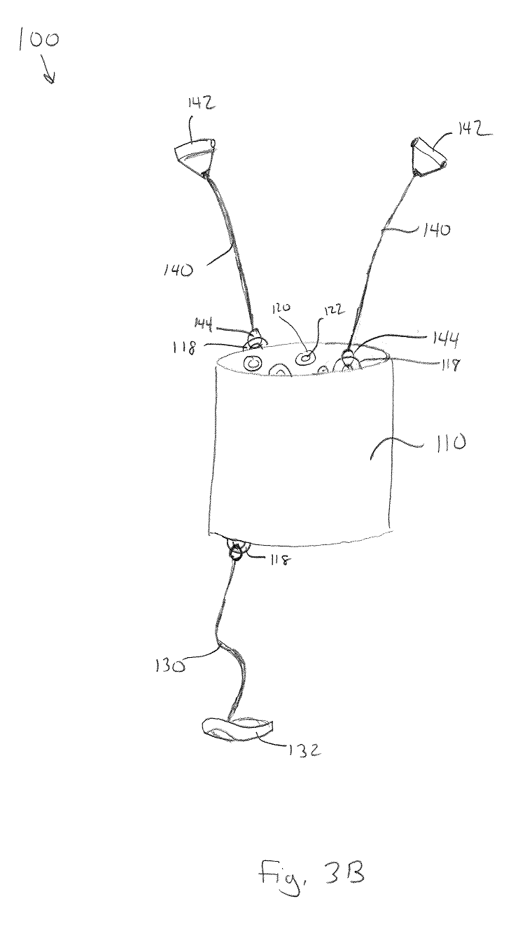

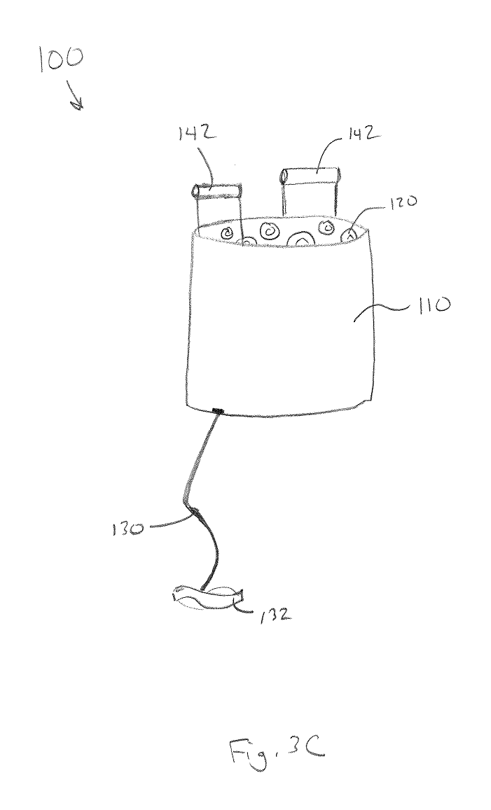

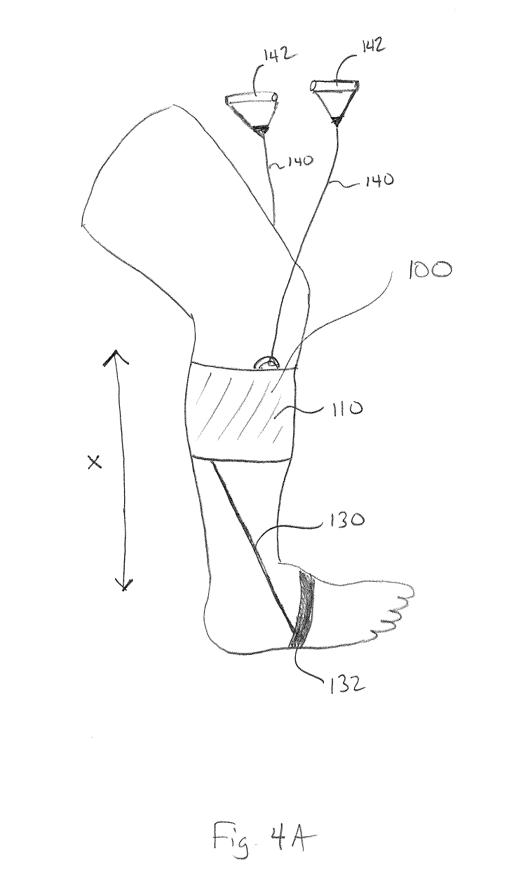

In some embodiments, the frame 110 further comprises one or more connectors 118 on the frame (see, FIGS. 1A through 1E). The connectors 118 can be used by the user of the device to physically move the frame 110 of the device 100 up and down the limb. In some embodiments, the device 100 comprises one or two handles 142 (see, FIGS. 3A through 3C). In some embodiments, the one or two handles 142 are directly connected to the frame 110 (see, FIG. 3C), thereby allowing the user to grasp the frame 110 directly (via the handles 142) and move the frame 110 about the limb while minimizing bending or stretching of the torso. In some embodiments, the one or two handles 142 are detachable. The one or more handles can be designed to be any shape or configuration. In some embodiments, a handle can be a knob, a grip, a ring, a bicycle pump-type handle, or the like.

The one or more handles can be attached or connected to the frame by any means. The one or more handles serve as a means for the user of the device 100 to move the device 100 along a limb. In some embodiments, the one or more (e.g., two) handles 142 are each connected to an upper leash 140, and each upper leash 140 is connected to the frame 110, such that the one or two handles 142 are indirectly connected to the frame 110 (see, FIGS. 3A and 3B). In some embodiments, the upper leash 140 can be integral with the frame 110 (see, FIG. 3A), or the upper leash 140 can be releasably connected to the frame 110, for example, via one or more ties 144, straps 144, clasps 144, clips 144, buckles 144, snaps 144, or hooks 144 that releasably latch or connect to the connector 118 on the frame 110 (see, FIG. 3B). Releasability allows different sized upper leashes 140 to be interchanged for use with the device 100. To move the frame about the limb, the user (or caretaker, trainer, therapist, practitioner, assistant, associate, etc.) pulls on the one or two handles 142, tensioning each upper leash 140, and thereby moving the frame 110 in the direction of the pull. In some embodiments, the upper leash 140 can be constructed such that the frame 110 can be pulled by the upper leash 140, for example, upward a limb, and the frame 110 can be pushed by the upper leash, for example, back down the limb, thus transversing the limb. In some embodiments, the upper leash 140 can be made to be retractable, such as in a typical handle of a suitcase.

In some embodiments, the device 100 can be tethered to the end of the limb (see, FIGS. 4A, 4B, and 4C). In some embodiments, the device 100 comprises one or more optional lower leashes 130 that can further include a patch 132 or strap 132 (see, FIGS. 3A through 3C). In some embodiments, the lower leash 130 can be tied or attached to an end of the limb, or the patch or strap 132 can be attached to an end of the limb (see, FIGS. 4A through 4C). For example, in some embodiments, the device 100 is connected to a lower leash 130 which is connected to a strap 132, such as a foot strap 132, that is attached to the foot of the user. In some embodiments, the lower leash 130 comprises an elastic material (e.g., natural or synthetic rubber, polymer, foam, nylon, latex, silicone, etc.) such that the lower leash 130 expands when the user pulls on the device 100 (e.g., pulls the handles 142 to move the frame), and then self-contracts when the user stops pulling on the device 100.

Elasticity of the lower leash 130 further facilitates therapeutic use of the device. By way of example, but not of limitation, the user may secure the strap 132 on their foot and secure the frame 110 on the lower portion of their calf. Then, the user pulls on the one or more handles 142 to move the frame 100 up the calf toward the knee causing the lower leash 130 to stretch. After the user stops pulling the frame 100 up their leg, and releases their pull tension, the lower leash 132 self-contracts, thereby pulling the frame 100 back down the leg toward the lower portion of the calf. The elasticity of the lower leash 132 helps to maximize user comfort, and to accommodate situations, for example, where the user is unable to bend over to move the frame 110 up and down the leg. Thus, the user can sit upright, stand, or lay down and comfortably move the device 100 up the limb by pulling the frame 110 upward, and allow the frame 110 to move by itself (via contraction of the elastic lower leash 132) down the limb, without the user having to bend to move the frame 110 down the limb. This is particularly advantageous in confined spaces such as in airplane seats or other vehicle, or for where the user has had surgery or an injury that limits their range of motion. Alternately, the frame 110 can be actuated by movement of the limb, for example, by the user holding the frame 110 steady/in place via the handles 142 and flexing or moving the limb such that the limb moves through the frame, rather than moving the frame 110 along the limb.

In some embodiments, the frame 110 is placed on the limb via the extremities. In some embodiments, the frame 110 is placed as a wrap, cup, or sleeve on the limb. For example, to place the frame 110 about the calf, the foot is first inserted into the tube and the frame 110 pulled up over the ankle and into place on the calf. Or for use on the arm, the hand is first inserted into the tube and the frame 110 is pulled up over the wrist and placed on the arm. In some embodiments, the frame 110 is open and then closed into a tube-like shape or sleeve-like shape or cup-like shape around the limb. For example, the frame 110 is wrapped around the limb, and then closed. In such embodiments, when the frame 110 is closed, it can form a tube-like shape or sleeve-like shape around the limb; in other embodiments, the closed frame 110 is not completely closed into a tube, but rather the two ends 111a and 111b of the partially closed frame 110 remain separated by a space (see, FIGS. 1A, 1B, and 1D). In some embodiments, the frame 110 is open and can be used to “cup” the limb, and held onto the limb or compressed closed by a user squeezing the ends 111a and 111b as close together as possible around the limb.

The frame 110 is intended to be able to move up and down a limb. A user controls this movement. The user may be the person on whom the frame 110 is attached, or may be an assistant to the person, such as a caretaker, trainer, therapist, medical practitioner, assistant, or associate. To facilitate movement, and also to further induce limb compression, the frame 110 comprises the plurality of compression units 120 described herein (see, FIGS. 2A through 2D).

Prevention or treatment of deep vein thrombosis, as well as the treatment of swelling associated with or without a deep vein thrombosis, with the device 100 involves movement of the frame 110 of the device 100 up and down an affected or at-risk limb (see, for example, FIGS. 4A, 4B, and 4C, axis x). The frame 110 compresses inward, and the compression units 120 further compress inward, and with compression of the limb provided and maintained by the device 100, the compression is moved along the limb as the user moves the frame 110. In some embodiments, the user engages the handles 142 or the handles 142/upper leash 140 system described herein to move the device up and down the limb. In some embodiments, the user engages the handles 142 or the handles 142/upper leash 140 system described herein to move the device up the limb, with the lower leash 130 and strap 132 providing the mechanism by which the device 100 returns to the original or lower position on the limb.

In some embodiments (not depicted in the drawings), the frame 110 locks onto a track (not shown) in which the track runs parallel to the limb guiding the frame 110 as it traverses the limb. In some embodiments, the track can be a two track system removably attached to the limb by, for example, VELCRO®. In some embodiments, one track is located on one side of the limb and the other track is located on the opposite side of the limb. In some embodiments, the track comprises a small motor or pneumatic activator allowing the movement of the frame 110 up and down the track, and controlled by the action of the user. In some embodiments, the frame 110 can comprise handles for allowing movement of frame 110 along the track manually. In some embodiments, the track comprises one or more locking mechanisms (not shown) to lock the frame 110 onto the track. In some embodiments, the locking mechanism comprises a latch, bolt, shackle, lever, cam, rim/mortise, or t-handle. In some embodiments, the user of the device 110 engages the motor or pneumatic activator, thus inducing movement of the device up the limb and down the limb. In some embodiments, the user can engage the pneumatic activator much like a bicycle pump handle to induce the movement up the track and up the limb. Upon reaching the top of the limb, the user, for example, can release the air in the pneumatic activator, thus allowing the device to return to the original or lower position on the limb. In some embodiments, the device is manually activated to sequentially compress from the bottom of the device (e.g., portion near terminal end of the limb such as the foot) to the top of the device (e.g., portion distal to the terminal end of the device such as the knee), thus forcing the flow of blood away from the terminal portion of the limb. Such activation can be carried out, for example, by a worm gear, or other such mechanism known to the skilled artisan.

The compression and movement of the device 100 helps to facilitate blood flow through the limb, preventing pooling and clotting of blood in the blood vessels (e.g., the deep veins) of the limb, and decreases swelling. The combined compression and movement provide the therapeutic benefit to the user. The embodiments of the device 100 described herein resulting in the ability to move the device 100 along the limb are only exemplary. Additional embodiments of the device 100 resulting in the ability to move the device 100 along the limb can be contemplated by the art skilled.

The present disclosure also provides methods for treating or preventing deep vein thrombosis, or deep vein thrombosis-associated swelling or other causes of limb swelling. In some embodiments, the methods comprise placing the frame 110 of the device 100 on a limb, thereby compressing the limb, and moving the frame 110 along the limb, thereby compressing different sections of the limb as the frame 110 is moved along the limb, such that deep vein thrombosis or deep vein thrombosis-associated swelling is prevented or treated. In some embodiments, the methods are employed on human subjects.

The present disclosure is not limited to the embodiments described and exemplified above, but is capable of variation and modification within the scope of the appended claims.

What is claimed is:

1. A device for compressing a limb, comprising:
a frame having an open configuration or a substantially tube-like or sleeve-like configuration;
a plurality of compression units on the internal surface of the frame; and
one or more handles connected to the frame, wherein the one or more handles is designed to move the frame along the limb.

2. The device according to claim 1, wherein the frame is unitary.

3. The device according to claim 1, wherein the frame comprises a plurality of segments operably connected to one or more pivotal linkages that allow the segments to move relative to each other and thereby allow the frame to expand and contract.

4. The method according to claim 3, wherein the one or more pivotal linkages comprises a reverse motion linkage, a push-pull linkage, a parallel motion linkage, a bell crank linkage, a crank and slider linkage, a worm gear, or a scissor linkage.

5. The method according to claim 3, wherein the one or more pivotal linkages further comprises a spring.

6. The method according to claim 3, wherein the one or more pivotal linkages comprises an elastic band.

7. The device according to claim 1, wherein the compression units comprise a ball, wheel, or roller.

8. The device according to claim 1, wherein the compression units further comprise a spring.

9. The device according to claim 1, wherein the device comprises one handle.

10. The device according to claim 1, wherein the device comprises two handles.

11. The device according to claim 1, wherein the one or more handles is connected to the frame by one or more upper leashes.

12. The device according to claim 1, wherein the one or more upper leashes is integral with the frame.

13. The device according to claim 1, wherein the one or more upper leashes is linked to a connector present on the frame.

14. The device according to claim 1, wherein the device further comprises a lower leash having elasticity that is connected at one end to the frame, and a patch or strap for tethering the frame to the limb that is connected to the other end of the lower leash.

15. The device according to claim 14, wherein the device comprises a foot strap.

16. A method of displacing fluid from and flow through a deep vein of a limb of a subject comprising:
attaching the device according to claim 1 to the limb, thereby compressing the limb; and
moving the device along the limb, thereby displacing fluid from and flow through the deep vein of the limb.

17. The method according to claim 16, wherein the user manipulates the one or more handles of the device to move the device along the limb.
